(19) United States
(12) Patent Application Publication
(10) Pub. No.: US 2017/0172728 A1
(43) Pub. Date: Jun. 22, 2017

(54) INTRAOCULAR LENS AND METHOD OF MANUFACTURING THE SAME

(71) Applicant: MAY-HWA ENTERPRISE CORPORATION, Taipei City (TW)

(72) Inventors: Hsien-Yeh Chen, Taipei City (TW); Shih-Kang Fan, Taipei City (TW); Jyun-Ting Wu, Taipei City (TW)

(21) Appl. No.: 14/997,591

(22) Filed: Jan. 18, 2016

(30) Foreign Application Priority Data
Dec. 22, 2015 (TW) 104143053

(51) Int. Cl. A61F 2/16

(52) U.S. Cl. CPC A61F 2/1613; A61F 2002/1683

(57) ABSTRACT

The present invention provides an intraocular lens, including a first poly-p-xylylene film, a second poly-p-xylylene film and a liquid drop. The liquid drop is disposed between the first poly-p-xylylene film and the second poly-p-xylylene. The present invention further provides a method of forming the same.

### BACKGROUND OF THE INVENTION

1. Field of the Invention

The present invention is related to an intraocular lens and a method of manufacturing the same, and more particularly, to an intraocular lens with a poly-p-xylylene film and a method of manufacturing the same.

2. Description of the Prior Art

Biomedical optics devices require specific parameters that meet both optical and bio-interfacial properties. The development of intraocular lenses (IOLs) has resulted in both research investigations and clinical products because of the enormous need to treat cataract patients (approximately 10 million IOLs are implanted yearly worldwide). In the search for relevant materials for fabricating IOLs, surface modifications on existing materials are currently being exploited; however, device associated problems, including postoperative calcification, dislocation, and the proliferation and migration of epithelial cells, which cause posterior capsular opacification or secondary cataracts, are lingering challenges. Some approaches solve one problem while compromising another issue, and current synthetic IOLs are still far from ideal.

Nevertheless, synthetic IOL products provide an effective solution for the immediate needs of cataract patients. In the design of future IOLs, it is desirable to obtain (i) enhanced compatibility with the environment of the posterior capsule in terms of position and chemical/biological properties, (ii) stability and durability to avoid leaching of potentially harmful substances to the surrounding biological environment, (iii) customizable optical and biological properties for diverse patient needs, and (iv) effective and simple procedures for delivery and implantation.

It is still a need to provide an intraocular lens which can meet the above requirements.

### SUMMARY OF THE INVENTION

The present invention therefore provides an intraocular lens and a method of forming the same, so as to meet the current requirements.

According to one embodiment, the present invention provides an intraocular lens, including a first poly-p-xylylene film, a second poly-p-xylylene film and a liquid drop. The liquid drop is disposed between the first poly-p-xylylene film and the second poly-p-xylylene.

According to another embodiment, the present invention further provides a method of forming an intraocular lens. First, a chemical vapor deposition (CVD) process is performed to form a first poly-p-xylylene film, following by placing a solution drop on the first poly-p-xylylene film. A chemical vapor deposition encapsulation process is performed to form a second poly-p-xylylene film on the first poly-p-xylylene film and the solution drop.

An innovative intraocular lens (IOL) device is fabricated based on a chemical vapor deposition encapsulation process using functionalized poly-p-xylylenes. The advanced IOL device provides noncompromised design parameters for both its optical and biological properties.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

FIG. 1 shows a flow chart of the method of manufacturing an intraocular lens according to one embodiment of the present invention.

FIG. 2 to FIG. 6 are schematic diagrams of the method of manufacturing an intraocular lens according to one embodiment of the present invention.

FIG. 7 is a schematic diagram illustrating a chemical vapor deposition system used in the present invention.

FIG. 8 and FIG. 9 show schematic diagrams of the method of manufacturing an intraocular lens according to another embodiment of the present invention.

FIG. 10 shows a schematic diagram of the method of manufacturing an intraocular lens according to another embodiment of the present invention.

FIG. 11 shows a schematic diagram of the method of manufacturing an intraocular lens according to another embodiment of the present invention.

FIG. 12 show photos and a bar chart with respect to the CA between the PPX film and silicone oil, PEG, 1,2,6-trihydroxyhexane, and glycerol, respectively.

FIG. 13 shows photos and a bar chart with respect to the CA between the PPX film and PEG, (PEG:glycerol)=1:1, (PEG:glycerol)=1:10, and glycerol, respectively.

FIG. 14 shows photos and a bar chart with respect to the CA between the PPX film with glycerol after treating plasma with argon, oxygen and C₄F₈, respectively.

FIG. 15 shows a line chart of the transmittance of the PPX-IOL with silicone oil, PEG, 1,2,6-trihydroxyhexane, and glycerol in the light of 250-800 nm

FIG. 16 shows comparison photos before and after the calcification treatment.

FIG. 17 are fluorescence micrographs showing (a) the enhanced cell adhesion and cell-resistant behaviors toward cultured HLECs and (b) moderate growth of HLECs is nonspecifically and homogeneously shown on the control surface of the unmodified PPX-IOL devices.

### DETAILED DESCRIPTION

To provide a better understanding of the present invention, preferred embodiments are detailed as follows. The preferred embodiments are also illustrated in the accompanying drawings to clarify the contents and effects of the present invention.

Please refer to FIG. 1, which shows a flow chart of the method of manufacturing an intraocular lens according to one embodiment of the present invention. As shown in FIG. 1, the method set forth in the present invention includes the following step:

Step 300: performing a chemical vapor deposition (CVD) process to form a first poly-p-xylylene film;

Step 302: placing a solution drop on the first poly-p-xylylene film; and

Step 304: performing a chemical vapor deposition encapsulation process to form a second poly-p-xylylene film on the first poly-p-xylylene film and the solution drop.

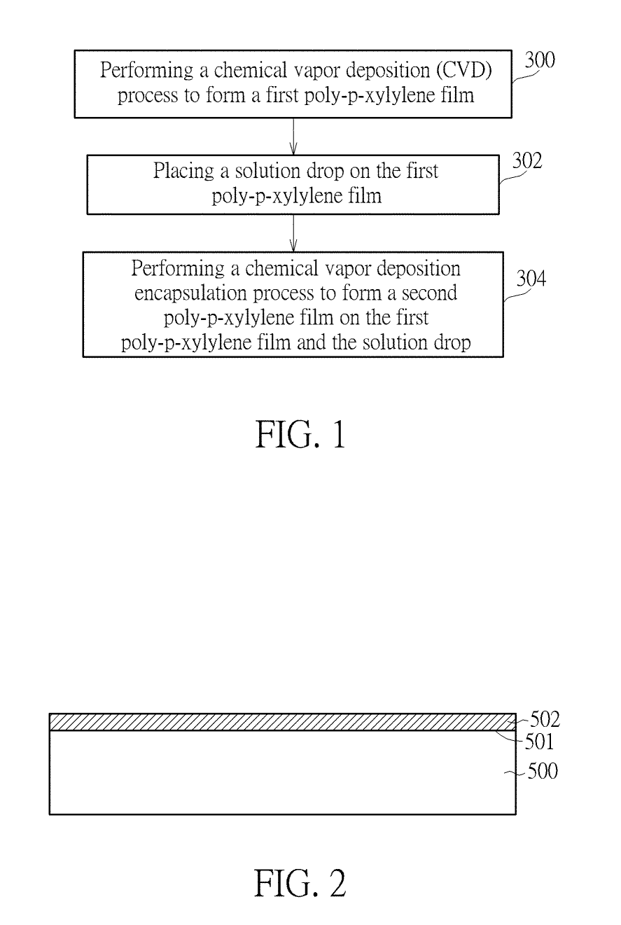

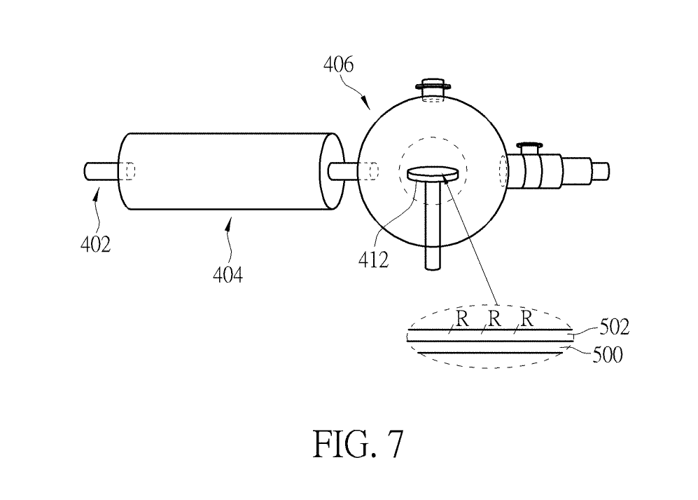

To clearly describe the above steps, please refer to FIG. 2 to FIG. 6 and FIG. 7, wherein FIG. 2 to FIG. 6 are schematic diagrams of the method of manufacturing an intraocular lens according to one embodiment of the present invention, and FIG. 7 is a schematic diagram illustrating a chemical vapor deposition system used in the present invention.

The method of manufacturing an intraocular lens, as shown in FIG. 2, begins by providing a substrate 500 and performing a chemical vapor deposition (CVD) process to form a first poly-p-xylene (PPX) film 502 on the substrate 500 (step 300). The substrate 500 can be any material capable of being used in the chemical vapor deposition process, such as a semiconductor, ceramics, glass, metal or any composition thereof. The semiconductor can be silicon or germanium. The glass can optionally be any doped glass or undoped glass. The metal can be copper (Cu), silver (Ag) or titanium (Ti), and can also be alloy, such as titanium alloy (Ti₆Al₄V). The composition can be any resin polymer, such as polystyrene (PS), or polymethylmethacrylate (PMMA). The substrate 500 can be a combination of the aforementioned materials, such as a silicon substrate having a silver film, but is not limited thereto. In another embodiment of the present invention, the substrate 500 can be a biological duct, stent, or pacemaker, but is not limited thereto. In one preferred embodiment, the substrate 500 is a SiO₂ substrate, having a surface 501. In this embodiment, the substrate 501 is a substantially flat surface. In another embodiment, the surface 501 can have other structures or devices, depending on the design of the product. Subsequently, a chemical deposition process is performed to form the first PPX film 502 directly on the surface 501 of the substrate 500. Specifically, the formed first PPX film 502 is formed by the CVD process with a pyrolysis process shown in below reaction 1, from as paracyclophane as a monomer.

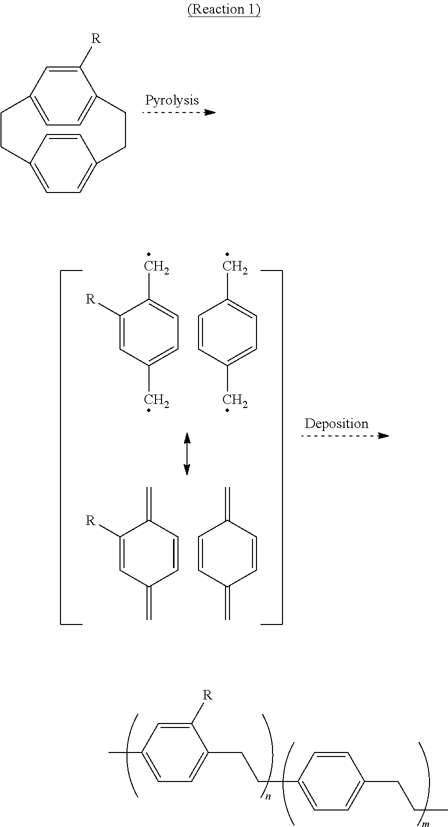

The paracyclophane in the present invention can have various functional group so as to form the functionalized first PPX film 502. In one embodiment, the first PPX film 502 includes the following structure with formula (1):

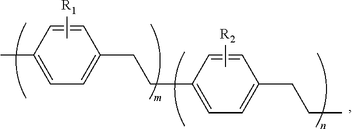

wherein R₁ and R₂ is selected from a group consisting of hydrogen —C(═O)H, —C(═O)—CFH₂, —C(═O)—CF₃, —C(═O)—C₂F₅, —C(═O)—C₈F₁₇, —C(═O)—OH, —C(═O)-Ph, —C≡CH, —CH═CH₂, —CH₂—OH, —CH₂—NH₂, —NH₂, —C(═O)—O—CH₃, —C(═O)—O—C₂H₅, —CH₂—O—C(═O)—C—(CH₃)₂Br, —CH₂—O—C(═O)—C≡CH, a chemical structure of formula (1-1), a chemical structure of formula (1-2) and a chemical structure of formula (1-3), and R₁ and R₂ are not simultaneous hydrogen, and m and n refer to an integral greater than 750,000:

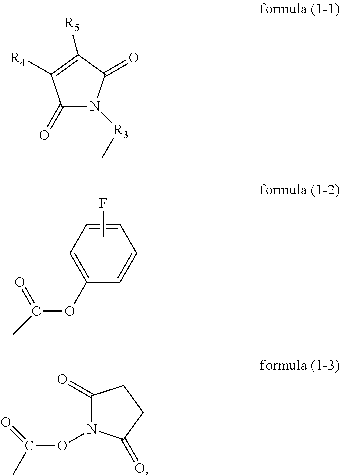

wherein in formula (1-1), R₃ refer to —CH₂—, —CH₂—CH₂—OC(═O)—, —CH₂—CH₂—NH—C(═O)—, —C(═O)— or —O—CH₂—; and R₄ and R₅ refer to hydrogen, methyl or chloride.

In another embodiment, the first PPX film 502 includes the following structure:

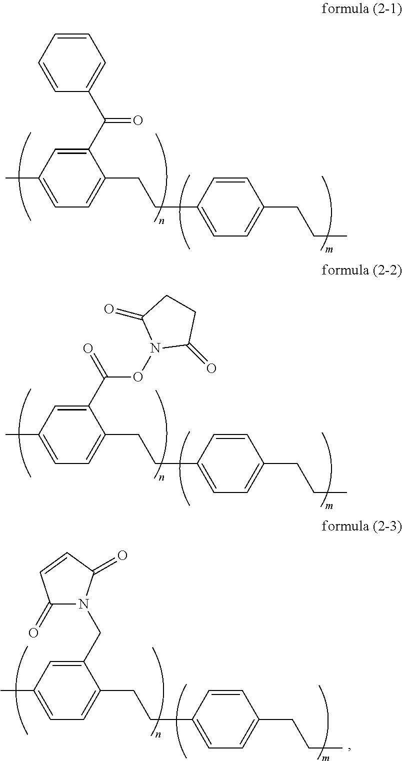

wherein m and n refer to an integral greater than 750,000.

In one preferred embodiment, the first PPX film 502 is a vinyl PPX film, in which the monomer thereof is 4-vinyl-[2,2]paracyclophane.

For the CVD process, please see FIG. 7. the chemical vapor deposition system 400 comprises a sublimation zone 402, a pyrolysis zone 404, and a deposition chamber 406. The paracyclophane monomer is inhaled from the sublimation zone 402, undergoes a pyrolysis process in the pyrolysis zone 404, and is then deposited on a substrate 500 placed on a supporter 412 in the deposition chamber 406. In one embodiment of the present invention, the chemical vapor deposition system 400 utilizes argon as the delivery gas to adjust systemic pressure, wherein the pressure of the chemical vapor deposition system 400 is adjusted under 100 mTorr, and the chamber is heated to 90° C. to prevent the monomer poly-p-xylylene from being deposited on the chamber. The sublimation temperature of the monomer is kept at 100 to 130 to 170 Celsius degrees, the sedimentation rate is adjusted to 1 Å/s via a quartz crystal microbalance (QCM), and the pyrolysis temperature is 510 to 800 Celsius degrees. The substrate 500 is placed on a supporter 412 having a room temperature, such as 20° C., wherein the supporter 412 is self-rotated to provide uniform coating of the first PPX film 502 on the substrate 500.

Subsequently, as shown in FIG. 3, after forming the first PPX film 502, an optional plasma treatment 520 is performed for the first PPX film 502, thereto adjust a surface wettability of the first PPX film 502. In one embodiment, the plasma treatment 520 is carried out via plasma source with a frequency during 10˜15 MHz that is used to discharge a gas containing argon, oxygen or C₄F₈ for treatment of the vinyl-PPX surfaces. In the plasma treatment 520, the pressure of the system is between 10⁻⁴ and 10⁻² torr, the gas flow is between 40 and 60 sccm, the power is between 10 W˜20 W, and the processing time is between 20 and 40 seconds.

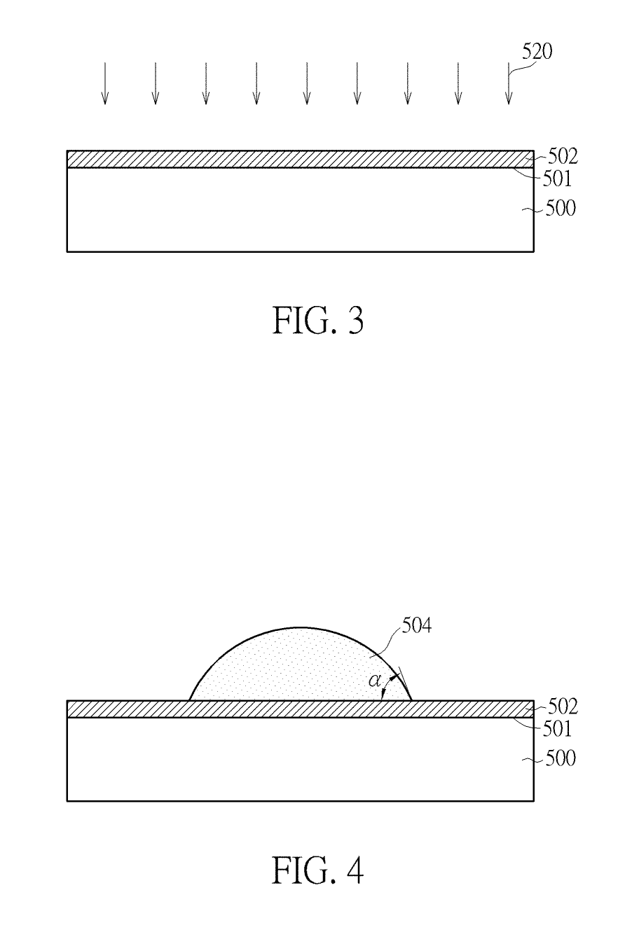

Next, as shown in FIG. 4, a solution drop 504 is formed on the first PPX film 502 (step 504). The solution drop 504 can be placed, for example by a dropper, to be formed directly on the first PPX film 502. One salient of the present invention is that a contact angle (CA) is formed between the solution drop 504 and the first PPX film 502, and the CA a can be adjusted according to the composition of the solution drop 504 and a wettability of the first poly-p-xylene film 502. In one embodiment, the solution drop 504 contains a solution having a vapor pressure under 0.1 mmHg at room temperature. For example, the solution drop 504 includes silicon oil, poly(ethylene glycol), 1,2,6-trihydroxyhexane or glycerol, and is not limited thereto. In one embodiment, the solution drop 504 includes a first solution and a second solution, in which the vapor pressure of the first solution and the vapor pressure of the second solution are both less than 0.1 mmHg. The first solution and the second solution are mixed in a predetermined ratio. In this manner, after placing the solution drop 504 directly on the first PPX film 502, a desired value of CA can be provided, without performing conventional electro-wetting process which uses additional voltage to alter the CA value. Thus, the substrate 500 in the present invention can use an insulation material such as SiO₂ or biocompatible material such as resin.

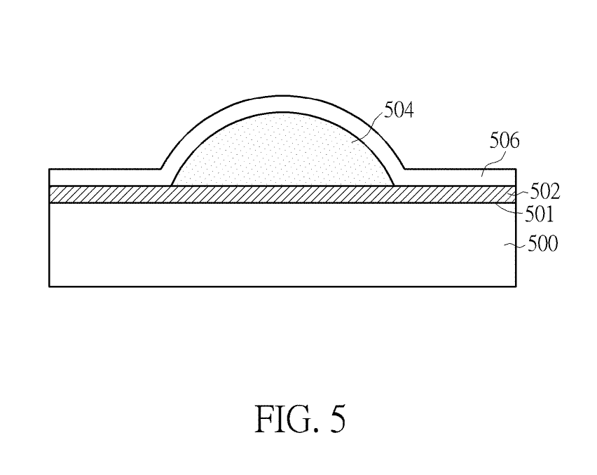

As shown in FIG. 5, a chemical vapor deposition encapsulation process is performed to form a second PPX film 506 on the first PPX film 502 and the solution drop 504 (step 304). In detail speaking, the second PPX film 506 is formed on the first PPX film 502 by a solid-on-liquid deposition to encapsulate the solution drop 504. The composition of the second PPX film 506 can be the same as that of the first PPX film 502 or they can be different so as to provide different on two sides of the intraocular lens. In one embodiment, the process of forming the second PPX film 504 is similar to the process of forming the first PPX film 502. Preferably, the supporter 412 of the chemical vapor deposition system 400 is maintained below the room temperature, such as less than 20 Celsius degrees, preferably less than 15 Celsius degrees, most preferably between −30 and −40 Celsius degrees, so as to prevent the solution drop 504 from evaporation.

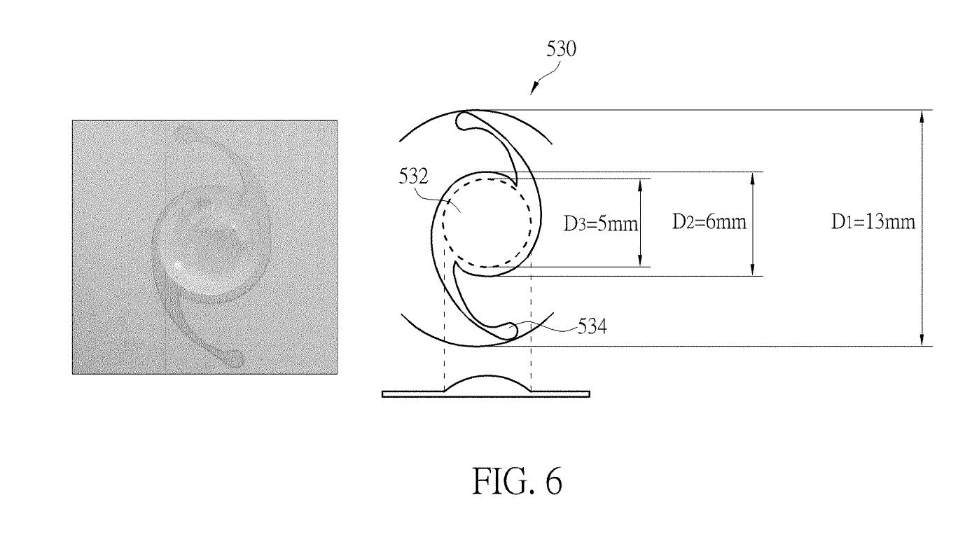

As shown in FIG. 6, a dicing process is carried out to form a desired shape of the intraocular lens (IOL) 530. The dicing process can be a laser process for example. The intraocular lens 530 can therefore be pick up from the substrate 500. As shown in the top view of FIG. 6, the IOL 530 has a 6 mm-diameter liquid optical region 532 and a pair of supporting haptic tails 534. The device was measured to be 13 mm long and 1 mm thick. After the above steps, the IOL of the present invention can be provided.

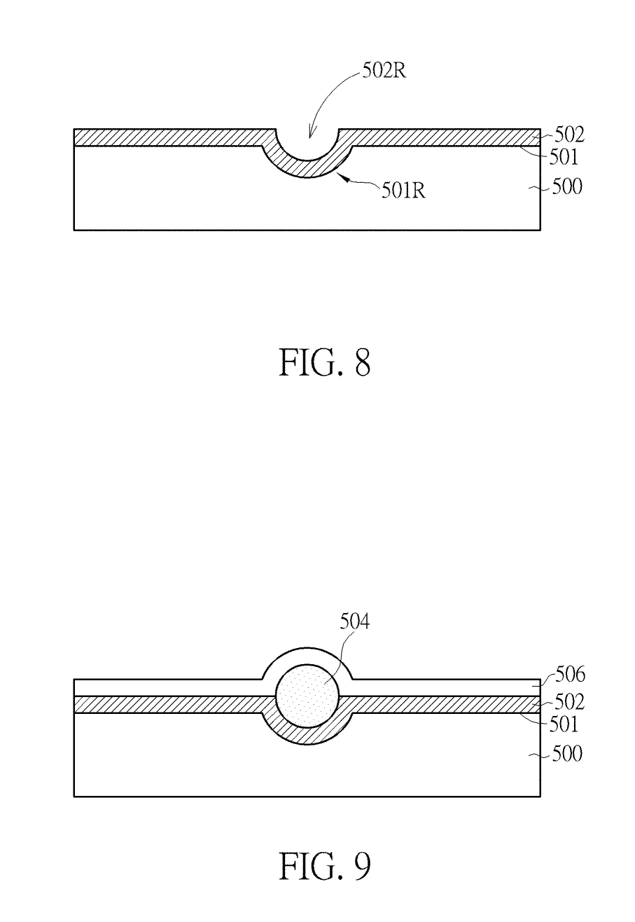

Please refer to FIG. 8 and FIG. 9, which show schematic diagrams of the method of manufacturing an intraocular lens according to another embodiment of the present invention. As show in FIG. 8, the surface 501 of the substrate 500 has a substrate recess 501R with a predetermined curvature. The formed first PPX film 502 will be formed conformally on the curved substrate recess 501R, so the formed first PPX film 502 has a film recess 502R. Next, as shown in FIG. 9, the solution drop 504 is formed on the first PPX film 502, preferably in the film recess 502R, and more preferably fitting the film recess 502R. Thereafter, the second PPX film 506 is formed on the solution drop 504 and the first PPX film 502. A dicing process is carried out to form the IOL 530. The IOL 530 of the present embodiment has two curved surfaces, wherein the curvature of one surface is decided by the substrate recess 501R (or the film recess 502R) and the curvature of the other surface is decided by the wettability of the first PPX film 502 and/or the composition of the solution drip 504. In another embodiment, the surface 501 of the substrate 500 can have other shapes, such as a mound shape, in order to form different types of IOLs 530.

Please refer to FIG. 10, which shows a schematic diagram of the method of manufacturing an intraocular lens according to another embodiment of the present invention. As shown in FIG. 10, after forming the second PPX film 506, a second solution drop 508 can be formed on the second PPX film 506. The embodiment of the second solution drop 508 can be similar to the solution drop 504, but can be adjusted according to the design of the product. Subsequently, a third PPX film 510 can be formed on the second solution drop 508 and the second PPX film 506. After the dicing process and removing it from the substrate 500, another embodiment of IOL can be provided.

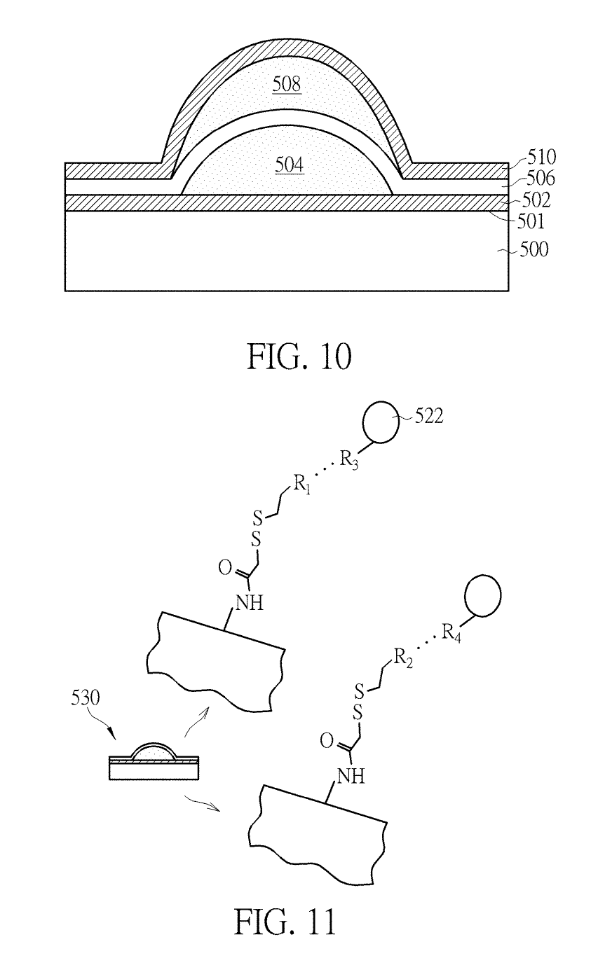

Please refer to FIG. 11, which shows a schematic diagram of the method of manufacturing an intraocular lens according to another embodiment of the present invention. As shown in FIG. 11, a treatment can be performed for anchoring a target molecule 522 onto an outer surface of the IOL 530 (such as the first PPX film 502, the second PPX film 506, the third PPX film 510, or their combinations). In one embodiment, the PPX film have disulfide bond and the target molecule 522 can be anchored onto the film by a thiol-ene interchange reaction. The target molecule 522 can be a pharmaceutical composition for treating eye disease. When the IOL 530 is implanted into the body, it can release the composition for treating eye disease such as cataract.

It is noted that the abovementioned embodiments of the IOL 530 can be combined arbitrarily to form various types of IOL 530. For example, the IOL with two curved surface shown in FIG. 9 can be incorporated into the IOL with multi-layered structure.

### Experiment 1 CA Value of the IOL Device

The shape and curvature of the PPX-IOL were controlled by varying the liquid wettability to produce varied optical properties. Prior to the encapsulation process, strategies for changing the liquid wettability were demonstrated by three different approaches: (i) by choosing liquids with varying wetting properties, (ii) by fine-tuning a mixture of two liquids with contrasting wettabilities, and (iii) by conducting plasma modification of the underlying surface wettability.

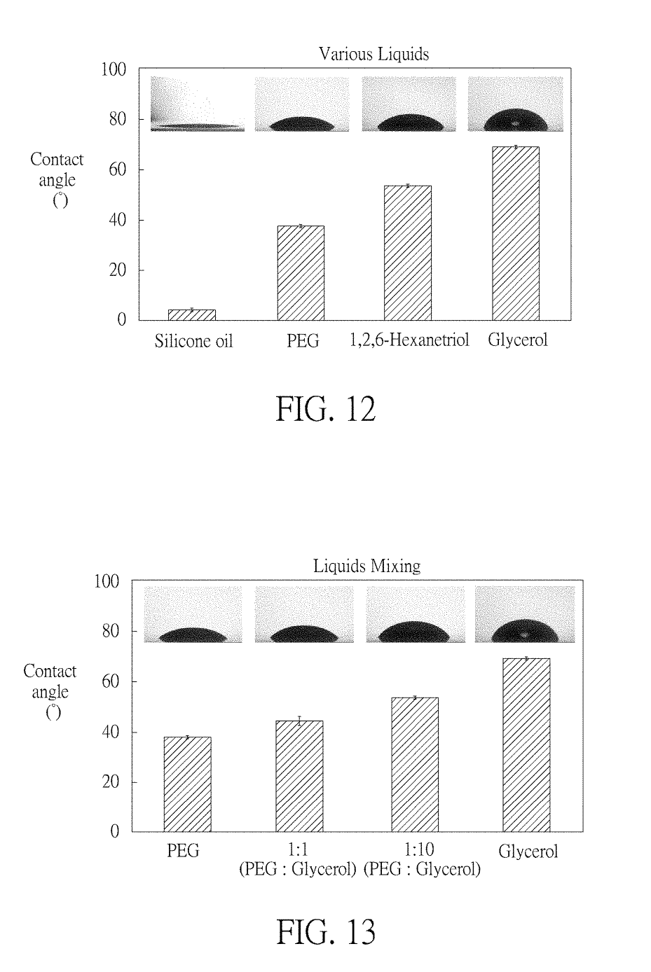

In the first approach, liquid droplets with a low vapor pressure, including silicone oil, poly(ethylene glycol) (PEG), 1,2,6-trihydroxyhexane, and glycerol, were placed on the previously deposited surface of the vinyl-PPX film. Prior to the encapsulation process, the wettability of each liquid was determined by placing a 2-μL droplet on the vinyl-PPX surface, and the static contact angle was measured by using a contact angle goniometer. As shown in FIG. 12, which shows photos and a bar chart with respect to the CA between the PPX film and silicone oil, PEG, 1,2,6-trihydroxyhexane, and glycerol, respectively. As shown in FIG. 12, the resulting contact angles (CAs) were measured as 4.63°±0.28°, 38.11°±0.46°, 53.85°±0.48°, and 69.23°±0.30°, respectively. These results indicate a wide range of wettability, and the desired wettability can be easily obtained by selecting a liquid from the list above or from other liquids

In the second approach, a mixture of two of the above liquids (if mixable) is created and the ratio is adjusted during mixing to form droplets with a tunable wettability. Please refer to FIG. 13, which shows photos and a bar chart with respect to the CA between the PPX film and PEG, (PEG:glycerol)=1:1, (PEG:glycerol)=1:10, and glycerol, respectively. As shown in FIG. 13, the CA values of (PEG:glycerol)=1:1, (PEG: glycerol)=1:10 are of 44.33°±1.37° and 54.34°±0.34°, respectively. The CA values are between 38.11°±0.46° (PEG) and 69.23°±0.30° (glycerol), as shown.

In the third approach, the plasma treatment with various gas for the supporting vinyl-PPX surfaces is performed. The plasma treatment was carried out via a radio frequency (13.56 MHz) plasma source that was used to discharge argon, oxygen, or C₄F₈ for treatment of the vinyl-PPX surfaces. The gas flow was 50 sccm for both argon and oxygen, and 50 sccm C₄F₈ was used together with 25 sccm of argon. A power of 15 W was maintained during the plasma process, and the processing time was 30 s for all plasma treatments. Please refer to FIG. 14, which shows photos and a bar chart with respect to the CA between the PPX film with glycerol after treating plasma with argon, oxygen and C₄F₈, respectively. As shown, the resulting surfaces exhibited varying wettabilities for the deposited glycerol droplets, with CA values of 20.95°±0.82°, 29.77°±1.29°, and 99.00°±0.40°.

### Experiment 2 Optical Characterizations

A summary of the effective focal lengths and refractive indices with respect to the liquid wettability is provided in Table 1. As shown in column 2 of Table 1, the refractive indices of the device range from 1.575 to 1.610. Though there is a great range of the wettability of the film, the variation of the refractive indices is small. High refractive indices were obtained for all of the combinations of PPX-IOL devices tested, which is attributed to the intrinsic index of refraction for vinyl-PPX (nD=1.611). Since the advanced PPX-IOL has a high refractive, the total volume of the solution drop can be lowered, and an ultra-thin PPX-IOX can be fabricated by using the chemical vapor deposition encapsulation process.

The effective focal lengths of PPX-IOL can be verified by using an OptiSpheric® instrument. As shown in column 3 of Table 1, the effective focal lengths of PPX-IOL can range from 4.394±01012 mm of C₄F₈ plasma treatment to >100 mm of silicon oil, showing a great tunable value. The corresponding changes in the effective focal length were confirmed to have a high dependency on the wetting properties of the encapsulated liquid. A low CA was correlated with a high focal length and vice versa. A desirable effective focal length can be obtained by fine-tuning the wettability.

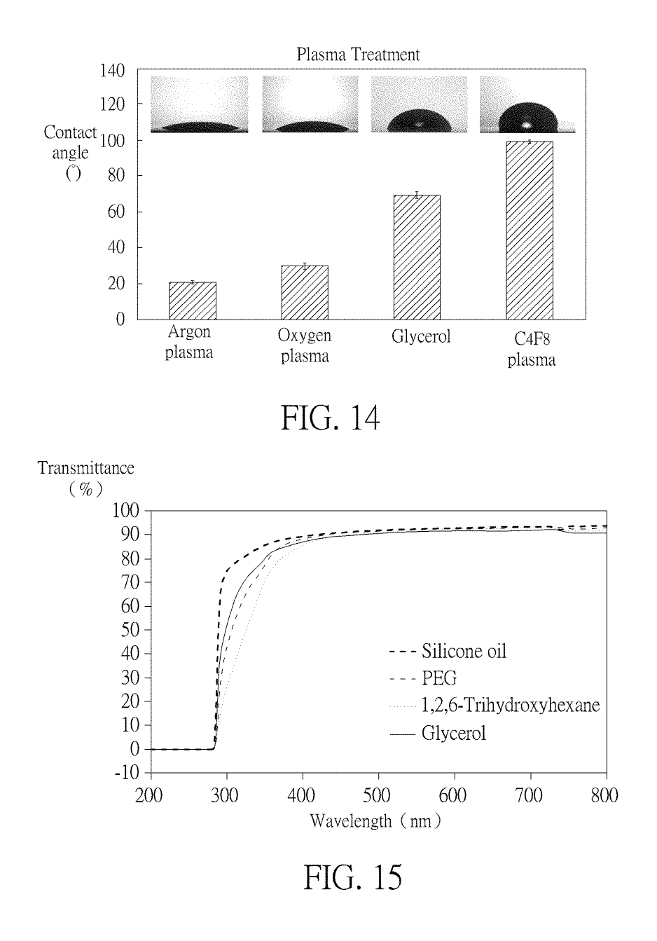

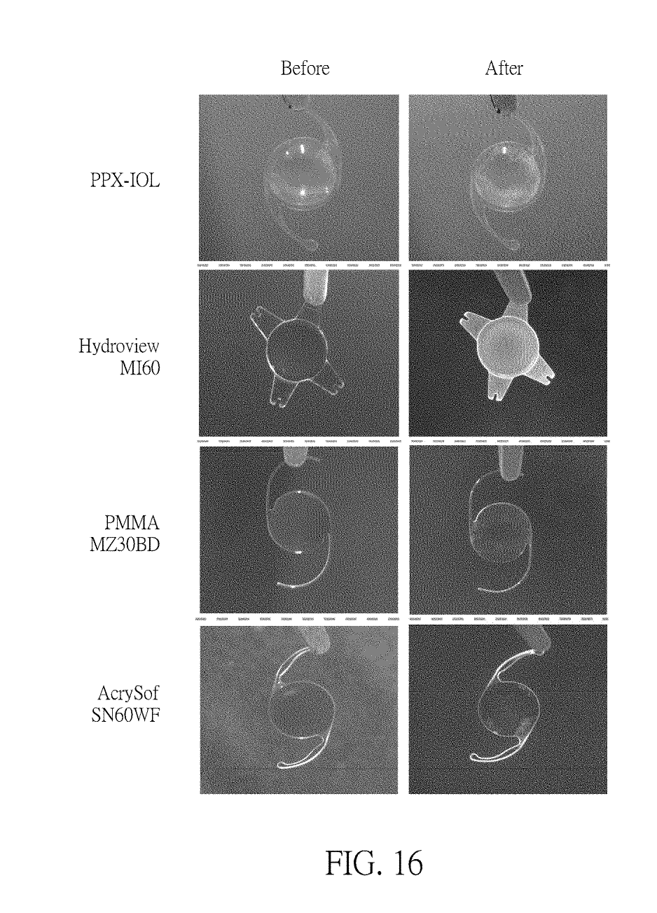

The optical properties were examined with respect to the transmittance of the PPX-IOL device by UV-vis analysis. Please refer to FIG. 15, which shows a line chart of the transmittance of the PPX-IOL with silicone oil, PEG, 1,2,6-trihydroxyhexane, and glycerol in the light range 250-800 nm. As shown in FIG. 15, the results indicated excellent transmission (>90%) of visible light (400-700 nm) for the devices. Strong absorption in the UV range (250-370 nm) was observed for all of the PPX-IOL, regardless of the encapsulated liquid, which is attributed to the inherent optical characteristics of vinyl-PPX. The result shows that the advance PPX-IOL can effectively resist the UV.

TABLE 1
refractive indices and effective focal lengths of encapsulated
liquid with varying wetting properties

| | Contact | Refractive | Effective |
|---|---|---|---|
| | angle | index | focal length |
| Liquid/Treatment | (degrees) | (—) | (mm) |
| Silicone oil | 4.63 ± 0.23 | 1.6074 ± 0.0011 | >100 |
| PEG | 38.11 ± 0.46 | 1.5688 ± 0.0006 | 10.695 ± 0.109 |
| 1,2,6- | 53.85 ± 0.48 | 1.6062 ± 0.0020 | 6.893 ± 0.014 |
| Trihydroxyhexane | | | |
| Glycerol | 69.23 ± 0.30 | 1.5890 ± 0.0017 | 5.965 ± 0.144 |
| PEG & glycerol/1:1 | 44.33 ± 1.37 | 1.5756 ± 0.0023 | 7.498 ± 0.192 |
| mixing | | | |
| PEG & | 54.34 ± 0.34 | 1.5835 ± 0.0011 | 6.579 ± 0.187 |
| glycerol/1:10 | | | |
| mixing | | | |
| Glycerol/Ar plasma | 20.95 ± 0.82 | 1.5960 ± 0.0015 | 28.607 ± 0.204 |
| treatment | | | |
| Glycerol/O₂ plasma | 29.77 ± 1.29 | 1.5981 ± 0.0006 | 24.755 ± 0.186 |
| treatment | | | |
| Glycerol/C₄F₈ | 99.00 ± 0.40 | 1.5787 ± 0.0013 | 4.394 ± 0.012 |
| plasma | | | |
| treatment | | | |

### Experiment 3 Calcification

The device/material-associated potency of calcium precipitation was examined in the PPX-IOLs. A simulated calcifying environment based on a highly concentrated calcium-potassium solution was used to examine the PPX-IOL, and control experiments were performed by comparing the IOLs with commercial IOLs, including Hydroview MI60 (Bausch & Lomb), PMMA MZ30BD (Alcon), and AcrySof SN60WF (Alcon) devices, which were all investigated in parallel. The IOL devices were immersed in a calcium-phosphate solution containing calcium chloride dihydrate, sodium phosphate monobasic monohydrate, and bovine serum albumin (BSA). Two calcifying solutions were prepared: one solution containing 100 mg/mL calcium chloride dehydrate, 100 mg/mL sodium phosphate monobasic monohydrate, and 200 mg/mL BSA and a second solution containing 200 mg/mL calcium chloride dehydrate, 50 mg/mL sodium phosphate monobasic monohydrate, and 200 mg/mL BSA. The IOL devices were alternately exposed to the two calcifying solutions (freshly prepared) every 2 days. The experiment was conducted at a constant temperature of 37° C. After 48 days of exposure, the samples were retrieved, washed with deionized water. Please refer to FIG. 16, which shows comparison photos before and after the calcification treatment. After 48 days of exposure to a calcifying environment, there was no sign of calcification for the PPX-IOL, as indicated by images obtained before and after the calcification test. Similarly, results of no calcification were also found for the control samples of PMMA MZ30BD and AcrySof SN60WF. In contrast, under the calcifying environment, Hydroview MI60 exhibited notable calcification. The result shows that the PPX-IOL is not prone to calcification.

### Experiment 4 Cell Attachment

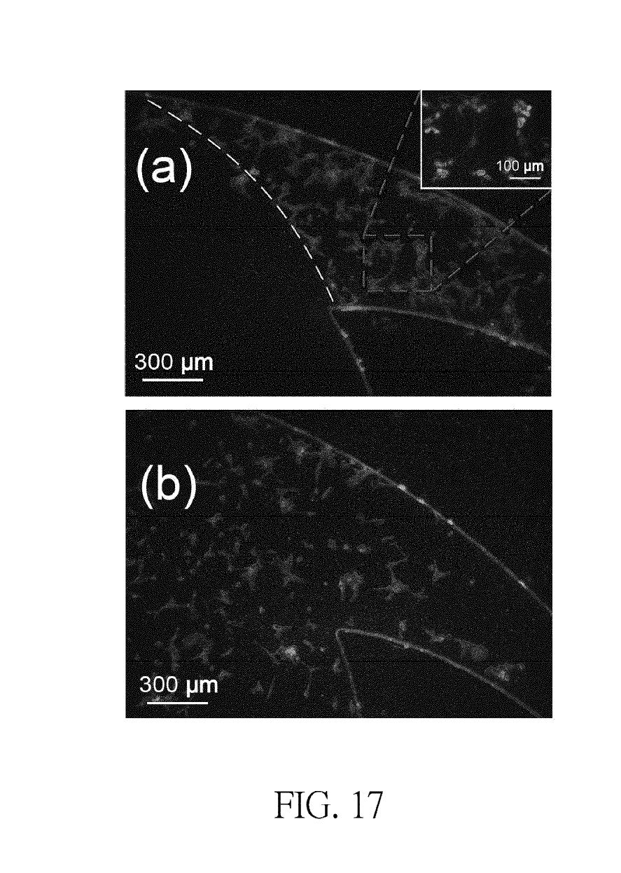

With respect to the surface chemical properties, the PPX-IOL provided additional ethylene anchoring sites to enable an orthogonal thiol-ene click reaction that can be activated photochemically. These anchoring sites were used to attach thiol-PEGs and cysteine containing peptides (Arg-Glu-Asp-Try-Try-Cys) (RGDYYC). The attachment of these molecules on selected areas is important in providing guided cell attachment cues for epithelial cells and is directed by a photoimmobilization procedure during the photochemically activated thiol-ene click reaction. Because of the curved surface of the IOL, a flat transparency photomask cannot be utilized for the photoimmobilization step. Instead, a microscopic patterning technique that is capable of precisely projecting desired patterns onto nonplanar surfaces was used for photoimmobilization of the IOL device. In this experiment, Human lens epithelial cells (HLECs) are seeded at a density of 1.5×10⁴ cells/cm² onto PPXIOL devices with previously immobilized thiol-PEG and RGDYYC. After a 24-h incubation, the resulting HLECs cells were fixed with 10% formalin, permeabilized with 0.1% Triton X-100 for 30 and 5 min, respectively, and then stained with 1 μg/mL 4′,6-diamidino-2-phenylindole and 50 μg/mL rhodamine-phalloidin for 15 and 30 min, respectively. The samples were then examined and photographed using a fluorescence microscope. Please refer to FIG. 17, which are fluorescence micrographs showing (a) the enhanced cell adhesion and cell-resistant behaviors toward cultured HLECs and (b) moderate growth of HLECs is nonspecifically and homogeneously shown on the control surface of the unmodified PPX-IOL devices. As shown in FIG. 17(a), HLECs are not attached to the central optical zone, proving the anti-cell attachment effect of the presence of PEG molecules. It is observed that HLECs are attached to the haptic tail region having modified RGD peptides, and a visible boundary is observed between the central optical region and haptic tail region. In contrast, in the IOL device without any modification, as shown in FIG. 17(b), HLECs discretely attached to the central optical zone and haptic tail region, showing that HLECs were allowed to grow nonspecifically and homogeneously on the control surfaces of unmodified PPX-IOL. Similar results are observed in other cell lines such as mouse embryonic fibroblasts (3T3) and corneal epithelial cells (HCECs).

In summary, an innovative intraocular lens (IOL) device was fabricated based on a chemical vapor deposition encapsulation process using functionalized poly-p-xylylenes. The advanced IOL device provides noncompromised design parameters for both its optical and biological properties. As an excellent optical device, it provides a high refractive index and a tunable effective focal length that is realized by manipulating the wetting properties of the encapsulated liquids; the device also offers protection from UV radiation. As a key medical device, it exhibits excellent biocompatibility and reduced postoperative calcification through the intrinsic properties of poly-p-xylylenes. In addition, these synergic functions are provided with precise surface chemistry for location to a guided attachment or repellent properties for eye epithelial cells, which is important in preventing device-associated complications.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. A method of manufacturing an intraocular lens, comprising:
performing a chemical vapor deposition (CVD) process to form a first poly-p-xylylene film;
placing a solution drop on the first poly-p-xylylene film; and
performing a chemical vapor deposition encapsulation process to form a second poly-p-xylylene film on the first poly-p-xylylene film and the solution drop.

2. The method of manufacturing an intraocular lens according to claim 1, wherein the substrate comprises an insulation material.

3. The method of manufacturing an intraocular lens according to claim 1, wherein the substrate comprises a surface and the first poly-p-xylylene film is formed directly on the surface of the substrate.

4. The method of manufacturing an intraocular lens according to claim 3, wherein the surface of the substrate is substantially a flat surface.

5. The method of manufacturing an intraocular lens according to claim 3, wherein the surface of the substrate comprises a recess.

6. The method of manufacturing an intraocular lens according to claim 1, wherein the first poly-p-xylylene film or the second poly-p-xylene film comprises the following structure: 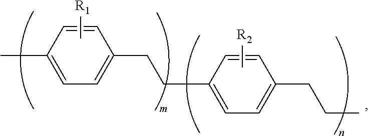
wherein R₁ and R₂ is selected from a group consisting of hydrogen —C(═O)H, —C(═O)—CFH₂, —C(═O)—CF₃, —C(═O)—C₂F₅, —C(═O)—C₈F₁₇, —C(═O)—OH, —C(═O)-Ph, —C≡CH, —CH═CH₂, —CH₂—OH, —CH₂—NH₂, —NH₂, —C(═O)—O—CH₃, —C(═O)—O—C₂H₅, —CH₂—O—C(═O)—C—(CH₃)₂Br, —CH₂—O—C(═O)—C≡CH, a chemical structure of formula (1-1), a chemical structure of formula (1-2) and a chemical structure of formula (1-3), and R₁ and R₂ are not simultaneous hydrogen, and m and n refer to an integral greater than 750,000: 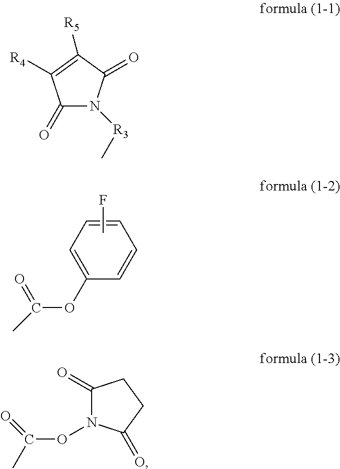
wherein in formula (1-1), R₃ refer to —CH₂—, —CH₂—CH₂—OC(═O)—, —CH₂—CH₂—NH—C(═O)—, —C(═O)— or —O—CH₂—; and R₄ and R₅ refer to hydrogen, methyl or chloride.

7. The method of manufacturing an intraocular lens according to claim 1, wherein the first poly-p-xylylene film or the second poly-p-xylene film comprises vinyl poly-p-xylylene.

8. The method of manufacturing an intraocular lens according to claim 1, wherein a contact angle is formed between the solution drop and the first poly-p-xylene film according to a composition of the solution drop and a wettablility of the first poly-p-xylene film.

9. The method of manufacturing an intraocular lens according to claim 1, wherein the solution drop comprises a first solution having a vapor pressure below 0.1 mmHg at room temperature.

10. The method of manufacturing an intraocular lens according to claim 9, wherein the first solution comprises silicon oil, poly(ethylene glycol), 1,2,6-trihydroxyhexane or glycerol.

11. The method of manufacturing an intraocular lens according to claim 1, wherein the solution drop comprises a first solution and a second solution, and a vapor pressure of the first solution is different from a vapor pressure of the second solution.

12. The method of manufacturing an intraocular lens according to claim 11, wherein the first solution or the second solution comprises silicon oil, poly(ethylene glycol), 1,2,6-trihydroxyhexane or glycerol.

13. The method of manufacturing an intraocular lens according to claim 1, further comprising:
before forming the solution drop, performing a plasma treatment for the first poly-p-xylylene film.

14. The method of manufacturing an intraocular lens according to claim 13, wherein the plasma treatment comprises supplying argon, oxygen or C₄F₈.

15. The method of manufacturing an intraocular lens according to claim 1, wherein in the chemical vapor deposition encapsulation process, the substrate is placed onto a supporter, and a temperature of the supporter is less than 20 Celsius degrees.

16. The method of manufacturing an intraocular lens according to claim 1, after forming the second poly-p-xylylene film, further comprising:
placing a second solution drop on the second poly-p-xylylene film; and
performing a second chemical vapor deposition encapsulation process to form a third poly-p-xylylene film on the second poly-p-xylylene film and the second solution drop.

17. The method of manufacturing an intraocular lens according to claim 1, further comprising:
after forming the second poly-p-xylylene film, anchoring a target molecule onto the first poly-p-xylylene film or the second poly-p-xylylene film.

18. The method of manufacturing an intraocular lens according to claim 17, wherein the target molecule comprises a pharmaceutical composition for treating an eye disease.

19. An intraocular lens, comprising:
a first poly-p-xylylene film;
a second poly-p-xylylene film; and
a solution drop disposed between the first poly-p-xylylene film and the second poly-p-xylene film.

20. The intraocular lens according to claim 19, wherein the first poly-p-xylylene film comprises an outer surface at the opposite side to the solution drop, wherein the outer surface is substantially a flat surface.

21. The intraocular lens according to claim 19, wherein the first poly-p-xylylene film comprises an outer surface at the opposite side to the solution drop, wherein the outer surface is curved.

22. The intraocular lens according to claim 19, wherein the first poly-p-xylylene film or the second poly-p-xylene film comprises the following structure: 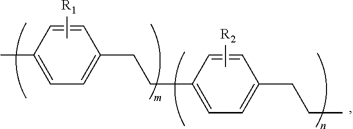
wherein R₁ and R₂ is selected from a group consisting of hydrogen —C(═O)H, —C(═O)—CFH₂, —C(═O)—CF₃, —C(═O)—C₂F₅, —C(═O)—C₈F₁₇, —C(═O)—OH, —C(═O)-Ph, —C≡CH, —CH═CH₂, —CH₂—OH, —CH₂—NH₂, —NH₂, —C(═O)—O—CH₃, —C(═O)—O—C₂H₅, —CH₂—O—C(═O)—C—(CH₃)₂Br, —CH₂—O—C(═O)—C≡CH, a chemical structure of formula (1-1), a chemical structure of formula (1-2) and a chemical structure of formula (1-3), and R1 and R2 are not simultaneous hydrogen, and m and n refer to an integral greater than 750,000: 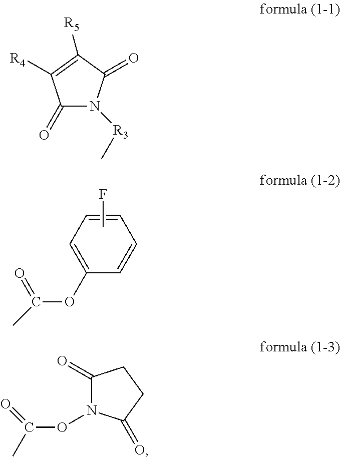
wherein in formula (1-1), R₃ refer to —CH₂—, —CH₂—CH₂—OC(═O)—, —CH₂—CH₂—NH—C(═O)—, —C(═O)— or —O—CH₂—; and R₄ and R₅ refer to hydrogen, methyl or chloride.

23. The intraocular lens according to claim 19, wherein the first poly-p-xylylene film or the second poly-p-xylene film comprises vinyl poly-p-xylylene.

24. The intraocular lens according to claim 19, wherein the solution drop comprises a first solution having a vapor pressure below 0.1 mmHg at room temperature.

25. The intraocular lens according to claim 24, wherein the first solution comprises silicon oil, poly(ethylene glycol), 1,2,6-trihydroxyhexane or glycerol.

26. The intraocular lens according to claim 19, wherein the solution drop comprises a first solution and a second solution, and a vapor pressure of the first solution is different from a vapor pressure of the second solution.

27. The intraocular lens according to claim 26, wherein the first solution or the second solution comprises silicon oil, poly(ethylene glycol), 1,2,6-trihydroxyhexane or glycerol.

28. The intraocular lens according to claim 19, further comprising:
a third poly-p-xylylene film, disposed on the second poly-p-xylylene film; and
a second solution drop, disposed between the second poly-p-xylylene film and the third poly-p-xylylene film.

29. The intraocular lens according to claim 19, wherein the first poly-p-xylylene film or the second poly-p-xylylene film anchors to a target molecule.

30. The intraocular lens according to claim 29, wherein the target molecule comprises a pharmaceutical composition for treating an eye disease.
